(19) United States
(12) Patent Application Publication
(10) Pub. No.: US 2025/0185936 A1
(43) Pub. Date: Jun. 12, 2025

(54) SYSTEMS AND METHODS FOR DETECTING MEDICAL CONDITIONS VIA ELECTROMAGNETIC FIELD DISTURBANCES

(71) Applicant: Paul C. Browne, Fripp Island, SC (US)

(72) Inventor: Paul C. Browne, Fripp Island, SC (US)

(21) Appl. No.: 18/887,568

(22) Filed: Sep. 17, 2024

Related U.S. Application Data
(60) Provisional application No. 63/607,889, filed on Dec. 8, 2023.

(51) Int. Cl. A61B 5/05; A61B 5/00

(52) U.S. Cl. CPC A61B 5/05; A61B 5/6803; A61B 5/6805; A61B 5/6823; A61B 5/6824; A61B 5/6828; A61B 5/6891; A61B 5/6892

(57) ABSTRACT

A system may detect a medical condition in a patient. The system may include an electromagnetic field generating unit, an electromagnetic field sensing unit, and a processing unit. The electromagnetic field generating unit may be operable to create an electromagnetic field about the patient. The electromagnetic field sensing unit may be operable to detect a disturbance in the electromagnetic field. The processing unit may be operable to process the disturbance in the electromagnetic field to extract a medical condition.

### CROSS-REFERENCE TO RELATED APPLICATIONS

This application claims priority to and benefit of U.S. provisional patent application No. 63/607,889 filed Dec. 8, 2023, which is herein incorporated by reference.

### TECHNICAL FIELD

The present disclosure generally relates to systems and methods for detecting medical conditions, and more particularly relates to systems and methods for detecting medical conditions via electromagnetic field disturbances.

### BACKGROUND

For the past 50 years, heart disease has been the most common cause of death in the United States. Of the 700,000 deaths from heart disease in the United States annually, the most common cause is congestive heart failure. The diagnosis of congestive heart failure relies on testing which documents heart injury from the disease. These technologies include X-ray, ultrasound, and mechanisms for measuring biomarkers in blood. The gold standard for the diagnosis of congestive heart failure is cardiac catheterization, which is an invasive procedure that increases the risk of stroke, heart attack, and death. The root cause of congestive heart failure is a decrease in energy production by heart muscle cells or the loss of heart cells from injury. None of the currently available diagnostic studies measure energy production by heart cells.

One type of monitor is a heart monitor, which monitors the emission of electromagnetic energy of the heart during heart contractions. The heart monitor provides real-time information regarding the emission of electromagnetic energy by the heart as the heart shrinks and rotates during a heartbeat. Other types of monitors might include a neurological monitor which measures electromagnetic energy produced by nerves in the brain and spine. Another type of monitor might include a muscle monitor which would measure the electromagnetic energy produced by somatic muscles when they contract. Other types of monitors can be envisioned that measure electromagnetic energy production by cells of the human body.

Electromagnetic energy is created by muscles and nerves at very low amplitudes. Direct measurements of the electromagnetic energy produced by the heart, for example, requires specialized, supercooled sensors. Electrocardiogram (EKG) monitors can qualitatively detect electromagnetic energy by using specialized electrodes attached to the skin. Electromagnetic energy produced by the brain and spine is also emitted at low amplitudes and is cyclic. Electroencephalogram (EEG) can qualitatively detect electromagnetic energy produced by the brain using specialized electrodes attached to the scalp. The measurement of electromagnetic energy produced by somatic muscles is also at low amplitudes and is difficult to measure secondary to motion. Electromyelogram (EMG) qualitatively detects electromagnetic energy produced by somatic muscles during muscle contractions using specialized electrodes attached to the skin. However, EKG, EEG, and EMG technologies do not measure the amount of electromagnetic energy produced by the heart, nerve cells and muscles. There are significant limitations in the current technology to directly measure electromagnetic energy produced by the heart, nerve cells and muscle cells.

### SUMMARY

A system may detect a heart condition in a patient. The system may include an electromagnetic field generating unit, an electromagnetic field sensing unit, and a processing unit. The electromagnetic field generating unit may be operable to create an electromagnetic field about the patient. The electromagnetic field sensing unit may be operable to detect a disturbance in the electromagnetic field. The processing unit may be operable to process the disturbance in the electromagnetic field to extract information which can be used to diagnose a medical condition.

A method may detect a medical condition via electromagnetic field disturbances. The method may include receiving information on the strength of the electromagnetic field, processing the electromagnetic field information and transmitting the information to one or more of the following: a display and a database.

Other systems, devices, methods, features, and advantages of the disclosed systems and methods will be apparent or will become apparent to one with skill in the art upon examination of the following figures and detailed description. All such additional systems, devices, methods, features, and advantages are intended to be included within the description and are intended to be protected by the accompanying claims.

### BRIEF DESCRIPTION OF THE FIGURES

The present disclosure may be better understood with reference to the following figures. Matching reference numerals designate corresponding parts throughout the figures, and components in the figures are not necessarily to scale.

FIG. 1 is a block diagram illustrating an embodiment of a system for detecting medical conditions via electromagnetic field disturbances.

FIG. 2 is a block diagram illustrating an embodiment of a generating unit operable to generate an electromagnetic field about a patient.

FIG. 3 is a partial cut-away, perspective view of an embodiment of a bed that has a number of electromagnets embedded therein.

FIG. 4 is a partial cut-away, perspective view of an embodiment of a mat that has a number of electromagnets embedded therein.

FIG. 5 is a partial cut-away, perspective view of an embodiment of a bed that has a number of electromagnets positioned about a lateral side of the bed.

FIG. 6 is a partial cut-away, perspective view of an embodiment of a chair that has a number of electromagnets positioned about the chair.

FIG. 7 is a partial cut-away, perspective view of an embodiment of a standalone device that has a number of electromagnets positioned about the standalone device.

FIG. 8 is a block diagram illustrating an embodiment of a sensing unit operable to detect a disturbance in an electromagnetic field.

FIG. 9a is a plan view of an embodiment of a vest device that is operable to detect a disturbance in an electromagnetic field, and FIG. 9b is a frontal view of the vest device positioned about the upper body of a patient.

FIG. 9c is a plan view of an embodiment of a cap device that is operable to detect a disturbance in an electromagnetic field, FIG. 9d is a frontal view of the cap device positioned on a head of a patient, and FIG. 9e is a rear view of the cap device positioned on a head of a patient.

FIG. 9f is a plan view of an embodiment of a sleeve device that is operable to detect a disturbance in an electromagnetic field, FIG. 9g is a side view of the sleeve device positioned about the arm of a patient, and FIG. 9h is a side view of the sleeve device positioned about the leg of a patient.

FIG. 10 is a schematic block diagram illustrating an embodiment of a device that is operable to detect a disturbance in an electromagnetic field.

FIG. 11 is a block diagram illustrating an embodiment of a method for detecting medical conditions via electromagnetic field disturbances, using a cardiac application.

### DETAILED DESCRIPTION

Described below are embodiments of a system and methods that permit detecting medical conditions (such as heart conditions, nerve conditions, muscle conditions, or any other types of medical conditions) via electromagnetic field disturbances. The systems and methods may create an electromagnetic field, may detect a disturbance in the electromagnetic field, and may process the disturbance to extract medical information, such as information about the functioning of a patient's organs, among other medical information. The systems and methods may detect medical conditions with relative specificity and sensitivity. The systems may create a method for the indirect measurement of electromagnetic energy created by the heart, nerves, and muscles.

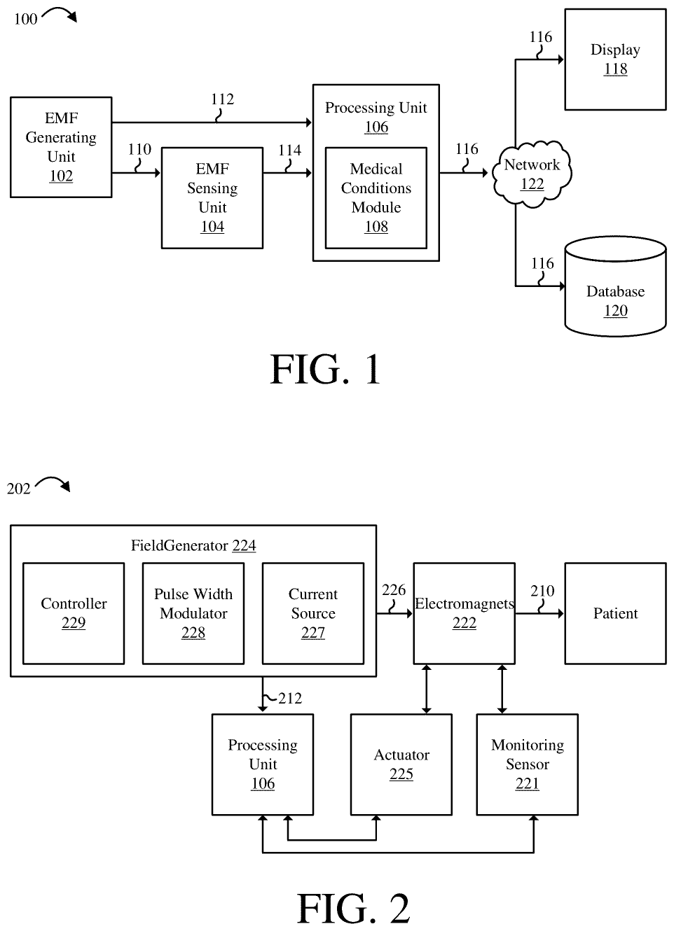

An embodiment of such a system 100 is shown schematically in FIG. 1. As shown, the system 100 generally includes a generating unit 102 operable to generate an electromagnetic field 110, a sensing unit 104 operable to detect a disturbance in the electromagnetic field 110, and a processing unit 106 operable to process disturbances in the electromagnetic field 110 to identify one or more medical conditions.

The EMF generating unit 102 may create an electromagnetic field 110 about a patient. The location of the electromagnetic field may depend on the medical condition desired to be monitored using the system 100. For example, if it is desired to obtain information about the functioning of a heart of the patient, then the electromagnetic field may be generated proximate to an upper body or a chest region of the patient. The electromagnetic field may also be generated about the head and spine, or in the region of somatic muscles of the arms, the legs, and the back. The electromagnetic field may be large enough to encompass the entire body of the patient in some circumstances. An example of the EMF generating unit 102 may include a field generator that causes one or more electromagnets to generate an electromagnetic field 110. The electromagnets may be positioned in any suitable arrangement, such as in a bed, in a chair, in a mat, on a side of a bed, on a standalone device, or in a combination of these and other locations. The EMF generating unit 102 may provide information 112 about the generated electromagnetic field 110 to the processing unit 106, which may facilitate identifying medical conditions.

The EMF sensing unit 104 may detect a disturbance in the electromagnetic field 110 caused by a heartbeat, a muscular contraction, and/or any other medical condition or combinations thereof. For example, the EMF sensing unit 104 may include a number of electromagnetic sensors. The sensors may be located in a vest device positioned about the patient, in a cap device provided on the head of the patient, in a knee sleeve or arm sleeve (or other type of exercise accessory) provided on the patient, or in a combination of these and other locations. The EMF sensing unit 104 may provide information 114 about disturbances in the electromagnetic field 110 to the processing unit 106, which may facilitate identifying medical conditions.

The processing unit 106 may include a medical conditions module 108, which may process the electromagnetic field information 112 and the disturbance information 114 to extract medical information 116. The medical information 116 may include Protected Health Information (PHI), patient demographics sufficient to identify a specific patient, or a combination thereof. The medical information 116 may be transmitted to a display 118 or database 120, such as via a network 122.

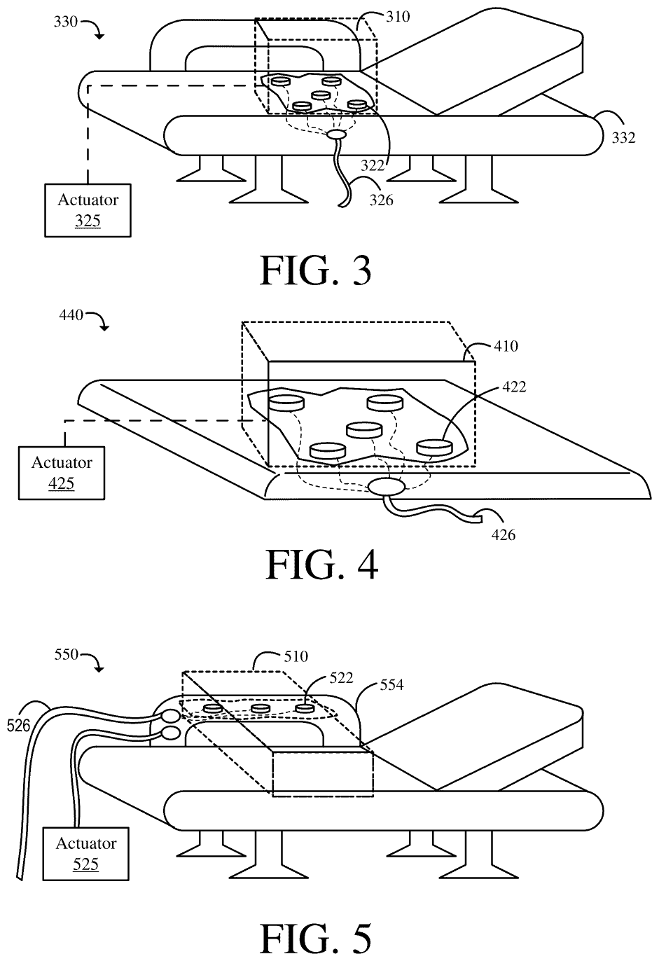

FIG. 2 is a block diagram illustrating an embodiment of an EMF generating unit 202. As shown, the EMF generating unit 202 may include one or more electromagnets 222 associated with a field generator 224. The electromagnets 222 may be positioned to produce an electromagnetic field 210 about the patient in a particular area of the patient depending on the type of medical condition that is desired to be monitored. For example, the electromagnets 222 may be positioned beneath the patient, above the patient, to a side of the patient, inside the patient, or a combination thereof. The field generator 224 may cause the electromagnets 222 to selectively produce the electromagnetic field 210, such as by supplying current to the electromagnets 222 via a field generator line 226. The resulting electromagnetic field 210 may be appropriate for the detection of medical conditions, including any medical conditions described herein or otherwise. For example, the electromagnetic field 210 may be sized to span the area of the upper body region of the patient if it is desired to obtain information about the functioning of the heart of the patient. Thus, a heartbeat may disturb the electromagnetic field 210 in a detectable manner. The strength of the electromagnetic field 210 may be sufficient to permit detecting medical conditions without adversely impacting the health of the patient. For example, the electromagnet field 210 may be a pulsed, low-frequency or extra-low frequency electromagnetic field. Further, each component of the EMF generating unit 202 may be sterilizable, disposal, or a combination thereof. In some embodiments, the electromagnets 222 may be positioned to produce an electromagnetic field 210 about the entire body of the patient.

In embodiments, the field generator 224 may provide current 226 to the electromagnets 222 through a field generator line 226, although other configurations are possible. The current 226 may be a constant current, an alternating current, a pulsed current, or some combination thereof. Depending on the current 226 supplied by the field generator 224, the resulting electromagnetic field 210 may be a constant field, a sinusoidally varying field, a pulsating field, or some combination thereof.

In embodiments, the field generator 224 may convert direct current to pulsed current for supplying to the electromagnets 222. An example of such an embodiment may include a direct current source 227 and a pulse width modulator 228. The pulse width modulator 228 may convert a direct current from the current source 227 to a pulsed current. Thus, the resulting electromagnetic field 210 may be pulsed intermittently, which may lower the overall strength of the electromagnetic field 210 without reducing the peak strength. For example, the electromagnetic field 210 may be a pulsed, low-frequency or extra-low frequency electromagnetic field.

In one example embodiment, the current source 227 may supply a current in the range from about less than one amp to about ten amps. The pulse width modulator 228 may pulse the current at a rate of about one hertz to about five megahertz. Upon application of such a pulsed current, the electromagnets 222 may produce an electromagnetic field 210 of about less than one watt per kilogram to about two watts per kilogram in 10 grams of tissue. Such an electromagnetic field 210 may be sufficient to detect medical conditions, even in obese patients, without harming the patient.

As an example, an electromagnetic field in the range of less than one to two watts per kilogram may be sufficient for a patient weighing between about 250 and about 300 pounds. Such an electromagnetic field may be generated using a current on the scale of about one amp and pulsed at about 0.1 to five megahertz. An electromagnetic field of about one watt per kilogram or less may be sufficient for a patient weighing about 150 pounds. Such an electromagnetic field may be generated using a current of less than about one amp and pulsed at about 0.1 to five megahertz.

In embodiments, the field generator 224 may be controlled by a controller 229. The controller 229 may control the current 226 in a manner that permits generating an electromagnetic field 210 that is of sufficient strength and penetration depth to detect the desired muscular contractions or emissions from nerves, yet is safe. For example, the controller 229 may control the frequency and voltage, which may manage the penetration of the electromagnetic field 210 through the patient and thus the amount of energy absorbed. The controller 229 also may operate the field generator 224 within a narrow range of current 226 to ensure a relatively uniform electromagnetic field 210 is generated.

In embodiments, the controller 229 may control the field generator 224 in response to one or more inputs. These inputs may be received from the processing unit 106 or from an input device or other user interface operated by a user, such as a keyboard or mouse. In response to the inputs, the controller 229 may control or provide instructions to one or more of the current source 227 and the pulse width modulator 228. For example, the electromagnetic field 210 may be adjusted in accordance with the weight of the patient, as a relatively stronger electromagnetic field 210 may facilitate detecting conditions in an obese patient. It should be noted that the controller 229 is shown as a component of the field generator 224 for functional purposes. In embodiments, the controller 229 actually may be a component of the processing unit 106, in which case the processing unit 106 may control the field generator 224.

In embodiments, the EMF generating unit 202 may include at least one actuator 225. The actuator 225 may be operable to actuate one or more of the electromagnet 222. For example, the actuator 225 may optimize the orientation or position of the electromagnets 222, such as by rotating or translating the electromagnets 222, to improve the accuracy or resolution of the system. For example, the actuator 225 may adjust the electromagnets 222 in accordance with instructions provided by the processing unit 106, which may be based at least in part on feedback regarding the electromagnetic field 210 provided by the EMF sensing unit. Thus, the electromagnetic sensor positions may be directed by the actuator 225 in the desired direction even as the patient moves and rotates.

The EMF generating unit 202 may provide electromagnetic field information 212 to the processing unit 106. For example, the electromagnetic field information 212 may be supplied by the field generator 224 via the controller 229. The electromagnetic field information 212 may include information about the current supplied by the current source 227, the frequency employed by the pulse width modulator 228, or the overall strength of the generated electromagnetic field 210, among other parameters or combinations thereof. The processing unit 106 may employ the electromagnetic field information 212 to adjust the settings of the controller 229, and to identify medical conditions, as described in further detail below.

In embodiments, the EMF generating unit 202 may also include one or more monitoring sensors 221. The monitoring sensors 221 may be operable to monitor the electromagnets 222. For example, the monitoring sensors 221 may be operable to measure the temperature of one or more of the electromagnets 222 and to communicate the measured temperature to the controller 229 or the processing unit 106. As another example, the monitoring sensors 221 may be operable to measure the current 226 received by the electromagnets 222 and to communicate the measured current to the controller 229 or processing unit 106. In the event an electromagnet 222 becomes overheated or receives excessive current, the controller 229 or processing unit 106 may interrupt or adjust the current source 227, the pulse width modulator 228, the field generator supply line 226, or a combination thereof. Thus, the electromagnet 222 may stop receiving current to ensure patient safety.

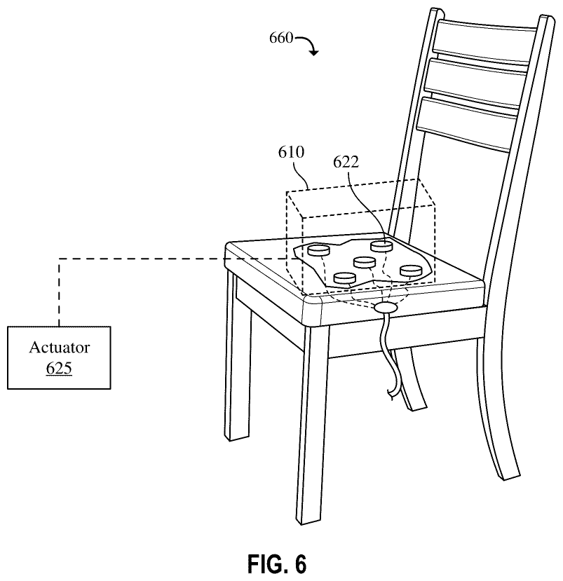

In embodiments, the electromagnets 222 may be selected, sized, and positioned to create an electromagnetic field 210 that substantially covers an area of the patient for which it is desired to obtain medical information (or the entire body of the patient). A single relatively large electromagnet 222 may be used, or an array of smaller electromagnets 222 may be used. It may be suitable to use an array of smaller electromagnets 222, as such electromagnets 222 may require relatively less current and may operate at relatively lower temperatures. Some electromagnets 222 may create an electromagnetic field 210 that propagates radially outward through an area that is relatively semi-spherical in shape, while other electromagnets 222 may create an electromagnetic field 210 that propagates relatively linearly through an area that is relatively columnar or beam shaped. It may suitable to use an electromagnet 222 that creates a columnar or beam-shaped electromagnetic field 210, as such electromagnets 222 may require relatively less current and may operate at relatively lower temperatures. Examples configurations for the electromagnets 222 are described below with reference to FIGS. 3-5.

Specifically, FIG. 3 is a perspective view of a bed 330 having a number of electromagnets 322 embedded therein. The bed 330 may be any type of hospital bed (or any other type of bed) now known or later developed. The electromagnets 322 may be embedded in a mattress 332 in an area directly below the area of interest of the patient. For example, if it is desired for the functioning of the heart of the patient to be determined, the electromagnets 322 may be embedded in an area below a chest or upper body region of the patient. The electromagnets 322 may also be positioned at any other location on and/or within the bed 330 as well (that is, the position of the electromagnets 322 shown in FIG. 3 is exemplary). Each electromagnet 322 may be in communication with a field generator via a field generator line 326. The electromagnets 322 may form an array. For example, the array may include between about two and about nine relatively smaller electromagnets. Other numbers and configurations may be used herein. Within the array, the electromagnets 322 may be arranged to create individual electromagnetic fields, each of which propagates upward through a relatively columnar area of the patient. One or more actuators 325 may actuate the electromagnets 322 so as to alter the direction of the electromagnetic field 310. The actuators 325 may actuate the electromagnets 322 individually or as a group. This will allow the electromagnetic field 310 to be focused on the area of interest of the patient. These electromagnetic fields 310 may combine to form an overall electromagnetic field that substantially covers the area of interest of the patient. Because the overall electromagnetic field 310 may substantially cover the area, the overall electromagnetic field 310 may be disturbed by muscular activity (contractions) or neurological activity within the area, facilitating detection. Because each individual electromagnet 322 may be relatively small, the electromagnets 322 may operate at relatively lower temperatures and may require less current. Examples of electromagnets 322 that may be used in such an array include Model E-16-260 Tubular Electromagnets manufactured by Magnetic Sensor Systems of Van Nuys, California, although any other suitable electromagnet may be used. Such electromagnets 322 may be relatively tubular in structure, resembling a hockey puck, although the electromagnets 322 may also have other shapes, such as square or rectangular shapes. The bed 330 may be suited for sterilization and re-use, and thus the bed 330 may protect the electromagnets 322.

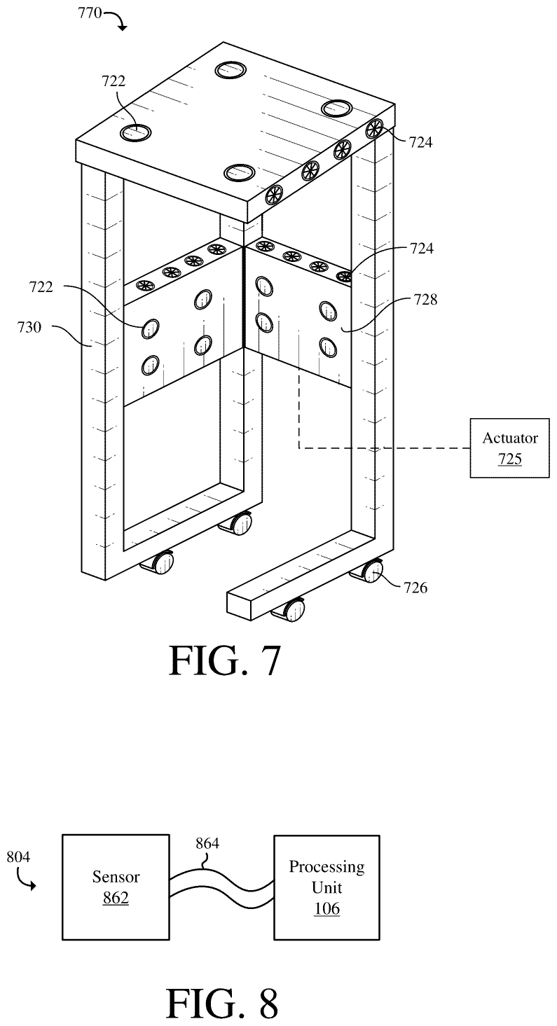

FIG. 4 is a partial cut-away, plan view a mat 440 having a number of electromagnets 422 positioned therein. The number and position of the electromagnets 422 shown in FIG. 4 is merely exemplary and any other configuration is possible. The mat 440 may be positioned under the patient, above the patient or to the side of the patient. For example, the mat 440 may be placed between the patient and the bed (however, the mat 440 may be provided at any other location, including separately from a bed as well). The mat 440 may be smaller than the bed, although any size is possible. The mat 440 may be manufactured of a biocompatible rubber material, such as a non-latex rubber. An example of such a rubber is ChronoPrene™ Thermoplastic Rubber Elastomer, which is made by AdvanSource Biomaterials Corporation of Wilmington, Massachusetts. Such a mat 440 may either be reusable or disposable. In embodiments in which the mat 440 is reusable, the mat 440 may be sterilizable. A disposable protective covering also may be positioned over the mat 440 to keep the mat 440 sterile. The cover may be formed from a material that does not attenuate the electromagnetic field, such as a plastic or impermeable cloth material. The mat 440 may also include a field generator line 426 that places the electromagnets 422 in communication with a field generator, such as the field generator 224.

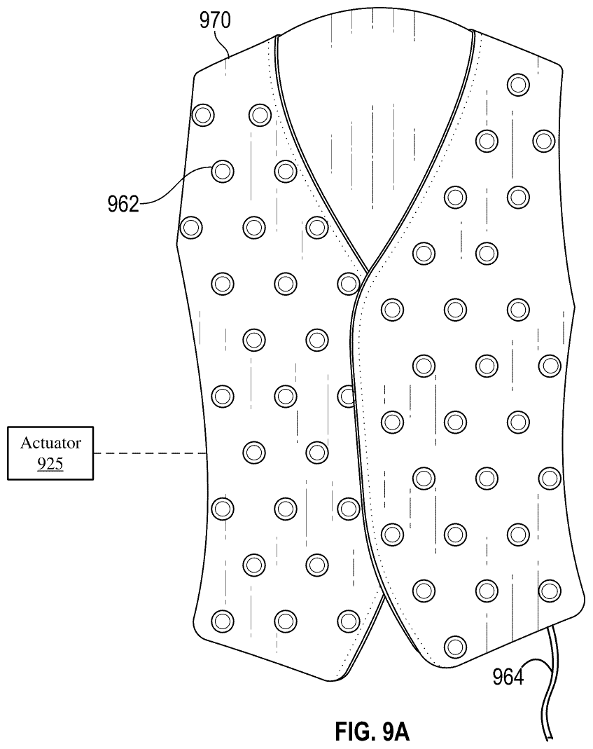

FIG. 5 is a perspective view of a bed 550 having a number of lateral electromagnets 522 positioned on a side of the bed 550. For example, the lateral electromagnets 522 may be associated with one or more bed rails 554, although other configurations are possible. The lateral electromagnets 522 may be in communication with a field generator, such as the field generator 224, via a field generator line 526. The lateral electromagnets 522 may be provided alone or in combination with electromagnets in a bed 330 or mat 440, as described above. In embodiments in which lateral electromagnets 522 are provided, these electromagnets 522 may create an electromagnetic field 510 that is generally transverse in direction to the field created by any electromagnets positioned under the patient. Thus, medical conditions may be detected with increased accuracy, as described in further detail below. The lateral electromagnets 522 also may be movable to accommodate movement of the patient. For example, an actuator 525 may move or rotate the electromagnets 522 in accordance with instructions provided by the processing unit 106, as described below. Thus, the electromagnets 522 may compensate for unanticipated movement or rotation of the patient.

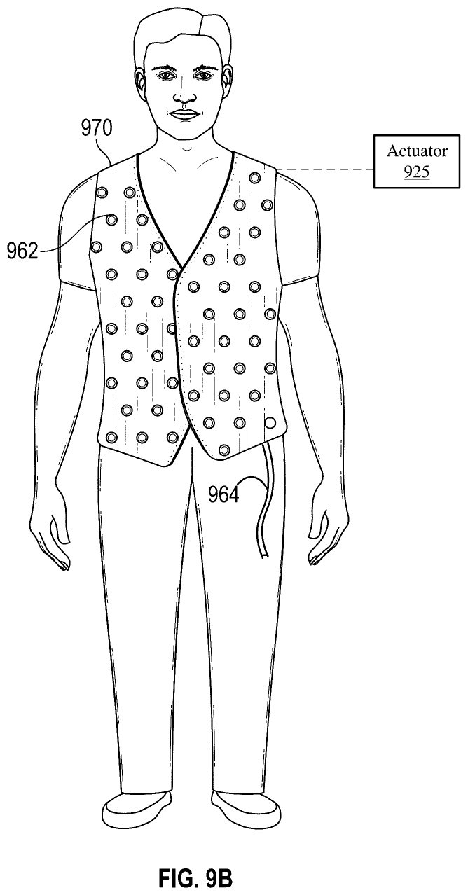

FIG. 6 is a perspective view of an embodiment of a chair 660 that has a number of electromagnets 622 positioned about the chair 660. The chair 660 may be any type of hospital chair (or any other type of chair) now known or later developed. Although a particular type of chair is depicted in the figure, this style of chair is not intended to be limiting. The electromagnets 622 may be embedded in a seat in an area directly below the area of interest of the patient. However, the electromagnets 622 may also be embedded in any other location within the chair as well, such as a back rest, one or more arms, and/or any other location or combination of locations. Each electromagnet 622 may be in communication with a field generator via a field generator line 626. The electromagnets 622 may form an array. For example, the array may include between about two and about nine relatively smaller electromagnets. Other numbers and configurations may be used herein. Within the array, the electromagnets 622 may be arranged to create individual electromagnetic fields, each of which propagates upward through a relatively columnar area of the patient. One or more actuators 625 may actuate the electromagnets 622 so as to alter the direction of the electromagnetic field 610. The actuators 625 may actuate the electromagnets 622 individually or as a group. This will allow the electromagnetic field 610 to be focused on the area of interest of the patient. These electromagnetic fields 610 may combine to form an overall electromagnetic field that substantially covers the area of interest of the patient. Because the overall electromagnetic field 610 may substantially cover the area, the overall electromagnetic field 610 may be disturbed by muscular activity (contractions) or neurological activity within the area, facilitating detection. Because each individual electromagnet 622 may be relatively small, the electromagnets 622 may operate at relatively lower temperatures and may require less current. Such electromagnets 622 may be relatively tubular in structure, resembling a hockey puck, although the electromagnets 622 may also have other shapes, such as square or rectangular shapes. The chair 660 may be suited for sterilization and re-use, and thus the chair 660 may protect the electromagnets 622.

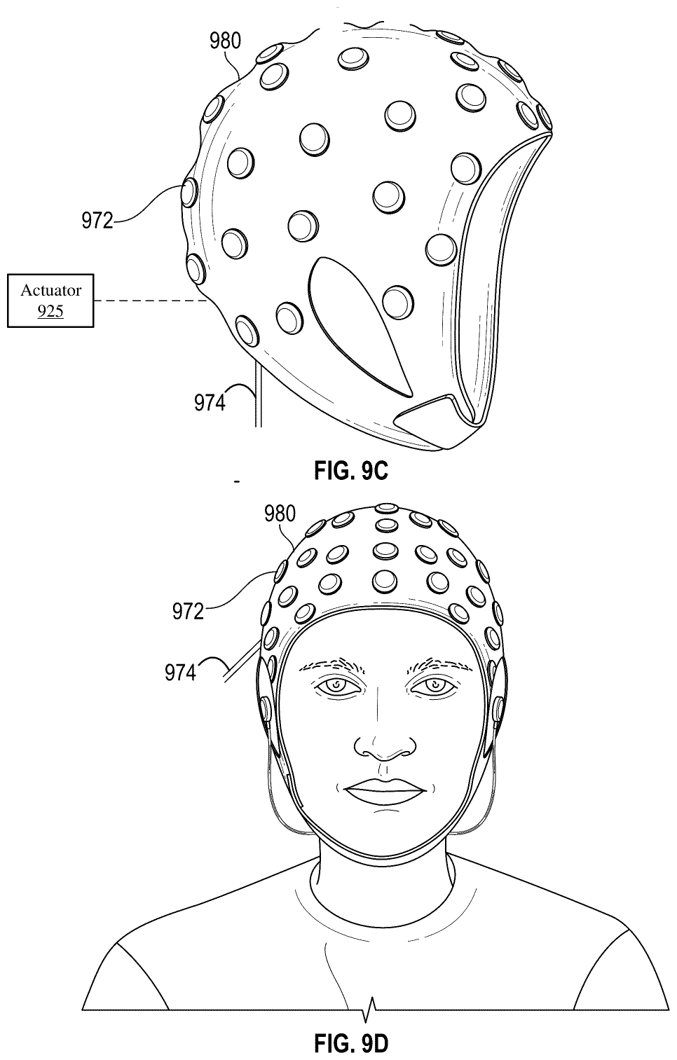

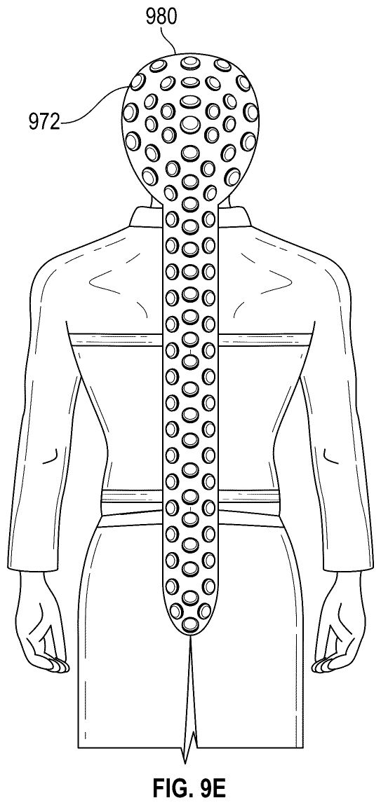

FIG. 7 is a perspective view of an embodiment of a standalone device 770 that has a number of electromagnets 722 positioned about the standalone device 770. The standalone device may include wheels 726 or other mechanisms that allow the standalone device 770 to be moved between various locations. For example, a medical professional may move the standalone device 770 to a location of a patient such that medical information about the patient may then be captured using the electromagnets 722. FIG. 7 shows that the electromagnets may be positioned at various locations along the standalone device 770, including, for example, above the patient, to the side of the patient, etc. The structures to which the electromagnets 722 are mounted may also be adjustable on the standalone device 722 such that the location of the electromagnets 722 may be adjusted relative to the portion of the patient that is desired to be monitored. As an example, side panels 728 and 730 may be adjustable up and down the standalone device 722. This adjustment may also be performed based on other factors, such as the height and weight of the patient, etc. However, the configuration of the standalone device 770 may also be fixed as well. The number and positioning of the electromagnets 722 shown in FIG. 7 is merely exemplary and other configurations are possible.

The standalone device 770 may also include one or more fans 724 that may provide cooling capabilities for the standalone device 770, including cooling of the electromagnets 722. Such fans 724 may also be provided on any other device described herein as well, such as the bed 330, mat 440, bed 550, chair 660, etc.

It should be noted that while FIGS. 3-7 show various types of EMF generating units (for example, the bed 330, the mat 440, the bed 550, the chair 660, and the standalone device 770), the EMF generating unit may also be provided in any other suitable form.

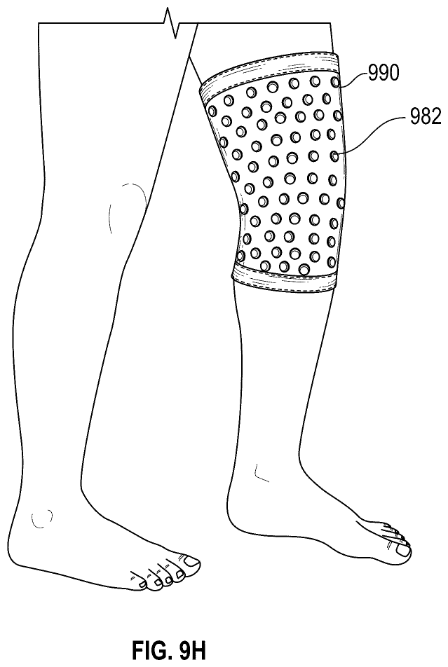

Returning to FIG. 1, the system 100 may also include the EMF sensing unit 104, which may be operable to detect a disturbance in the electromagnetic field 110 and to communicate information 114 about the disturbance to the processing unit 106. FIG. 10 is a block diagram illustrating an embodiment of such an EMF sensing unit 1004. As shown, the EMF sensing unit 1004 may include one or more electromagnetic sensors 1062 and a disturbance information line 1064. The electromagnetic sensors 1062 may be positioned about or within the patient. The electromagnetic sensors 1062 may detect disturbances in the electromagnetic field that are caused by muscular contractions or neurological activity in the area through which the electromagnetic field propagates. The disturbance information line 1064 may communicate information about the disturbances from the sensors 1062 to the processing unit 106, which may process the disturbance information as described below.

In embodiments, the EMF sensing unit 1004 may cooperate with the processing unit 106 to control one or more of the electromagnets. With reference back to FIGS. 2-7, the EMF generating unit may include an actuator operable to adjust the orientation or position of electromagnets. The EMF sensing unit 1004 may provide feedback to the processing unit 106 that permits optimizing the orientation or position of the electromagnets to improve the accuracy or resolution of the system. In turn, the processing unit 106 may provide instructions to the actuator, which may adjust the electromagnets accordingly. Thus, the electromagnetic sensors 1062 may be able to continuously detect the electromagnetic field generated by electromagnets even as the patient moves and rotates.

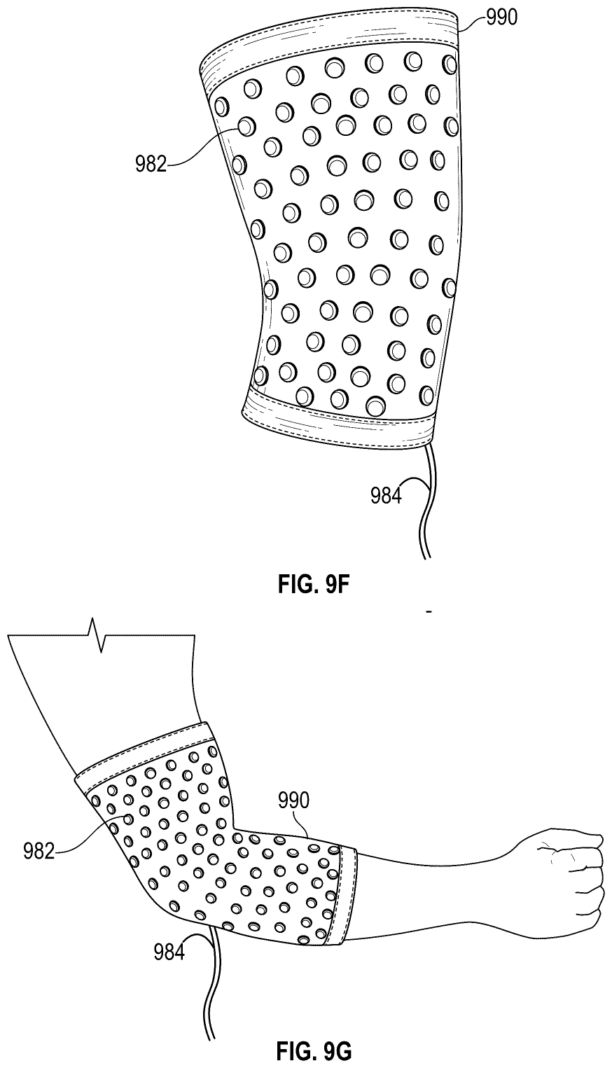

FIG. 9a is a view of an embodiment of an EMF sensing unit 1004 that includes a vest device 970, and FIG. 9b is a side view of the vest device 970 positioned about the upper body of a patient. The vest device 970 may have one or more sensors 962 in communication with a disturbance information line 964 suited for communicating disturbance information to the processing unit 106. The vest device 970 be formed from a biocompatible, pliant, and comfortable material, such as an elastic polymer material or a spandex material. The material may be double-layered so that the sensors 962 may be held out of contact from the patient. The dimensions of the vest device 970 may depend on the size of the patient. For example, the vest device 970 may be configured to be worn by a patient with a bodily circumference between about 18 inches and about 100 inches, although other circumferences are possible. To ensure a suitably sized vest device 970 is available, the vest device 970 may be made in different sizes, the vest device 970 may be adjustable, or a combination thereof. The vest device 970 also may have a closure or fastener that facilitates attaching the vest device 970 about the patient. Example closures or fasteners include velcro, snaps, buttons, hooks and loops, or other suitable closures. Once the vest device 970 is positioned about the patient, the vest device 970 may form to the contour of the patient and the sensors 962 may become appropriately positioned to detect disturbances in the electromagnetic field. After use, the vest device 970 may be either reused or disposed. If reusable, the vest device 970 may be designed for sterilization between uses, or may be covered in a disposable protective cover. Such a protective cover may be formed from a material that does not attenuate the electromagnetic field.

Each sensor 962 may be operable to detect disturbances in the electromagnetic field and to communicate disturbance information to the processing unit 106 through the disturbance information line 964. The disturbance information may be in a variety of forms depending on the configuration of the sensors 962. For example, the sensors 962 may measure the strength of the electromagnetic field and may output a measurable parameter, such as a voltage, in proportion to the measured strength. Example sensors include MC95Rw sensors which are manufactured by Magnetic Sciences in Acton, Massachusetts, although any other suitable sensors may be used.

In embodiments, the vest device 970 may include an array of sensors 962. The sensors 962 may be positioned within the vest device 970 so that when the vest device 970 is attached to the patient, the array covers a suitable portion of the upper body of the patient. As shown in the illustrated embodiment, the array may include a number of sensors 962 arranged in various rows at various intervals. In other embodiments, other configurations are possible. For example, the number of sensors 962 may be adjusted depending on the size of the patient.

Each sensor 962 may have a designated location within the vest device 970, and this location may be known by or communicated to the processing unit 106. The sensors may detect the electromagnetic field in more than one axis. Information from the electromagnetic field sensors may include axis information or other information. Information from the sensor array may be used to identify the location, strength, direction of propagation, duration and other information from disturbances in the electromagnetic field. The processing unit 106 may use the designated location of the sensor(s) when processing the disturbance information to identify the medical condition(s).

In other embodiments, one or more internal sensors positioned within certain regions of the body of the patient, which may increase the sensitivity of detection. Thus, the internal sensor may be relatively smaller than an external sensor without decreasing effectiveness. The internal sensors may be particularly relevant for obese patients, as external sensors positioned about an obese patient may be relatively farther from the internal organs and other internal portions of the patient than comparable external sensors positioned about a thinner patient. The internal sensor also may facilitate distinguishing the source of the disturbance in the electromagnetic field, due to the close proximity to the monitored sites.

FIG. 9c is a view of an embodiment of an EMF sensing unit 1004 that includes a cap device 980, FIG. 9d is a frontal view of the cap device 980 positioned about a head of the patient, and FIG. 9e is a rear view of the cap device 980 positioned about a head of the patient. The cap device 980 may have one or more sensors 972 in communication with a disturbance information line 974 suited for communicating disturbance information to the processing unit 106. The cap device 980 be formed from a biocompatible, pliant, and comfortable material, such as an elastic polymer material or a spandex material. The material may be double-layered so that the sensors 972 may be held out of contact from the patient. The dimensions of the cap device 980 may depend on the head size of the patient. To ensure a suitably sized cap device 980 is available, the cap device 980 may be made in different sizes, the cap device 980 may be adjustable, or a combination thereof. Once the cap device 980 is positioned on the head of the patient, the cap device 980 may form to the contour of the patient and the sensors 972 may become appropriately positioned to detect disturbances in the electromagnetic field. After use, the cap device 980 may be either reused or disposed. If reusable, the cap device 980 may be designed for sterilization between uses, or may be covered in a disposable protective cover. Such a protective cover may be formed from a material that does not attenuate the electromagnetic field.

Each sensor 972 may be operable to detect disturbances in the electromagnetic field and to communicate disturbance information to the processing unit 106 through the disturbance information line 974. The disturbance information may be in a variety of forms depending on the configuration of the sensors 972. For example, the sensors 972 may measure the strength of the electromagnetic field and may output a measurable parameter, such as a voltage, in proportion to the measured strength.

In embodiments, the cap device 980 may include an array of sensors 972. The sensors 972 may be positioned within the cap device 980 so that when the cap device 980 is attached to the patient, the array covers a suitable portion of the head of the patient. In some embodiments, the number of sensors 972 may be adjusted depending on the size of the patient. In embodiments, a set of sensors may be provided on the cap device 980 that extends from the back of the head to the tip of the spine (or any other distance down the patient's body).

Each sensor 972 may have a designated location within the cap device 980, and this location may be known by or communicated to the processing unit 106. The sensors may detect the electromagnetic field in more than one axis. Information from the electromagnetic field sensors may include axis information or other information. Information from the sensor array may be used to identify the location, strength, direction of propagation, duration and other information from disturbances in the electromagnetic field. The processing unit 106 may use the designated location of the sensor(s) when processing the disturbance information to identify the heart conditions (or other conditions), as described below. The position of the sensors 972 may be adjusted by an actuator 925 based on information provided to the processing unit 106.

As shown in FIG. 9e, the sensors of the cap may extend down the neck and back of the patient such that sensors may be positioned over a spine or similar region of the patient. The sensors may extend down the neck and back of the patient any other length as well.

FIG. 9f is a view of an embodiment of an EMF sensing unit 1004 that includes a sleeve device 990, FIG. 9g is a view of the sleeve device 990 positioned about an arm of the patient, and FIG. 9h is a view of the sleeve device 990 positioned about a leg of the patient.

The sleeve device 990 may have one or more sensors 982 in communication with a disturbance information line 984 suited for communicating disturbance information to the processing unit 106. The sleeve device 990 be formed from a biocompatible, pliant, and comfortable material, such as an elastic polymer material or a spandex material. The material may be double-layered so that the sensors 982 may be held out of contact from the patient. The dimensions of the sleeve device 990 may depend on the size of the patient and/or the area of the body of the patient on which the sleeve is worn. For example, a sleeve configured for use on an arm of the patient may be smaller than a sleeve configured for use on a leg of a patient. However, the sleeve may be sufficiently stretchable such that the sleeve may be used on either the arm or leg of the patient. To ensure a suitably sized sleeve device 990 is available, the sleeve device 990 may be made in different sizes, the sleeve device 990 may be adjustable, or a combination thereof. The sleeve device 990 also may have a closure or fastener that facilitates attaching the sleeve device 990 about the patient. Example closures or fasteners include velcro, snaps, buttons, hooks and loops, or other suitable closures. However, in some embodiments, the sleeve device 990 may also not include a closed sleeve that is not able to be opened and closed using a closure or fastener. For example, the sleeve device 990 in such embodiments may be pulled over the arm or leg of the patient (or any other portion of the body of the patient). Once the sleeve device 990 is positioned about the patient, the sleeve device 990 may form to the contour of the patient and the sensors 982 may become appropriately positioned to detect disturbances in the electromagnetic field. After use, the sleeve device 990 may be either reused or disposed. If reusable, the sleeve device 990 may be designed for sterilization between uses, or may be covered in a disposable protective cover. Such a protective cover may be formed from a material that does not attenuate the electromagnetic field. The position of the sensors 982 may be adjusted by an actuator 925 based on information provided to the controller 106.

Each sensor 982 may be operable to detect disturbances in the electromagnetic field and to communicate disturbance information to the processing unit 106 through the disturbance information line 984. The disturbance information may be in a variety of forms depending on the configuration of the sensors 982. For example, the sensors 982 may measure the strength of the electromagnetic field and may output a measurable parameter, such as a voltage, in proportion to the measured strength. Example sensors include MC95Rw sensors which are manufactured by Magnetic Sciences in Acton, Massachusetts, although any other suitable sensors may be used.

In embodiments, the sleeve device 990 may include an array of sensors 982. The sensors 982 may be positioned within the sleeve device 990 so that when the sleeve device 990 is attached to the patient, the array covers a suitable portion of an arm or a leg of the patient (or any other portion of the body of the patient). The number of sensors 982 may be adjusted depending on the size of the patient.

Positioning the sensors 982 in an array may facilitate differentiating contractions of muscular contractions of the patient. For example, the sleeve device 990 may be worn by the patient during exercise and information about the patient may be obtained as the patient performs physical exercises. For example, if the patient is using the sleeve device 990 during a personal therapy session for a leg injury, the sleeve device 990 may be worn over a leg of the patient to capture data about the leg as the patient undergoes physical therapy using the leg, which may include leg exercises. Each sensor 982 may have a designated location within the sleeve device 990, and this location may be known by or communicated to the processing unit 106. The sensors may detect the electromagnetic field in more than one axis. Information from the electromagnetic field sensors may include axis information or other information. Information from the sensor array may be used to identify the location, strength, direction of propagation, duration and other information from disturbances in the electromagnetic field. The processing unit 106 may use the designated location of the sensor(s) when processing the disturbance information to identify the heart conditions (or other conditions), as described below.

While reference is made herein to a sleeve device 990, any other suitable type of device may also be used. For example, a belt device or abdominal binder may be provided around an abdominal region of the patient, etc.

FIG. 10 is a block diagram that schematically illustrates an embodiment of an EMF sensing unit 1004 for positioning within a device of the patient. The EMF sensing unit 1004 may include a device 1080 and one or more internal sensors 1062 positioned on the device 1080. The device 1080 may be associated with a disturbance information line 1064 that communicates disturbance information from the internal sensor 1062 to the processing unit 106.

Returning to FIG. 1, the system may include a processing unit 106, which may include a medical conditions module or engine 108. The medical conditions module or engine 108 may receive the electromagnetic field information 112 and the disturbance information 114. The medical conditions module or engine 108 may process this information 112, 114 to extract medical information 116. The medical information 116 may include information about the functioning of a heart of the patient and/or any other types of medical information described herein or otherwise. The medical conditions module or engine 108 may transmit the medical information 116 to a display 118, a database 120, or a combination thereof.

In embodiments, the processing unit 106 may be operable to receive one or more user inputs. For example, the processing unit 106 may be in communication with an input device, such as a user interface, that permits a user to enter information such as the weight of the patient, existing heart conditions or other medical conditions, or other information. The processing unit 106 may employ such user input information to identify medical information 116.

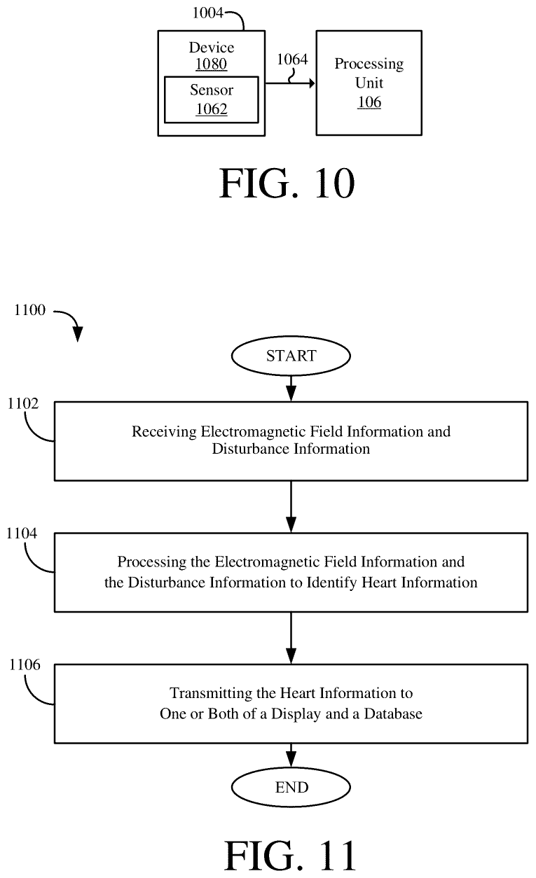

More specifically, the medical conditions module or engine 108 may execute a method 1100 for identifying medical information via electromagnetic field disturbances. An embodiment of such a method is illustrated in FIG. 11.

In block 1102, electromagnetic field information 112 and disturbance information 114 are received. The electromagnetic field information 112 may be received from the EMF generating unit 102, while the disturbance information 114 may be received from the EMF sensing unit 104. The electromagnetic field information 112 may correlate to the electromagnetic field provided to the chest or upper body region of the patient (or any other region of the patient's body). The disturbance information 114 may correlate to the electromagnetic field detected in the upper body region of the patient (or any other region of the patient's body). In some embodiments, user input information may also be received.

In block 1104, the electromagnetic field information 112 and the disturbance information 114 may be processed to extract medical information 116. The medical information 116 may include any medical information, such as information associated with the functioning of the heart of the patient and/or any other information described herein or otherwise. In some embodiments, the user input information may be processed along with the electromagnetic field and disturbance information 112, 114.

In embodiments, processing the electromagnetic field information 112 and the disturbance information 114 in block 1004 may include identifying a cause of a disturbance in the electromagnetic field. When a muscular contraction occurs in the abdominal area, a disturbance in the electromagnetic field may result. The disturbances may be revealed upon comparing the provided electromagnetic field to the detected electromagnetic field, as indicated by the electromagnetic field information 112 and the disturbance information 114 respectively. The disturbances may be further analyzed to identify the cause. For example, the disturbance may have one or more disturbance characteristics that facilitate identifying the cause. The disturbance characteristics may include a magnitude of the disturbance, a duration of the disturbance, a location of the disturbance, a cycling or repetition of the disturbance, or a combination thereof. It is noted that information about the location of the disturbance may be available in embodiments in which the EMF sensing unit 104 includes an array of sensors. In such cases, the disparate locations of the sensors in the array may facilitate obtaining disturbance location information.

The disturbance characteristics may be modeled to identify trends or patterns that indicate the cause. For example, information obtained by the sensor array in a patient undergoing a cardiac examination may indicate a decrease in the electromagnetic energy emitted by the heart. Such a decrease may be associated with a medical condition such as congestive heart failure or cardiomyopathy. Disturbance characteristics may follow these and other trends or patterns, facilitating the identification of the cause of the disturbance.

In embodiments, processing the electromagnetic field information 112 and the disturbance information 114 in block 1104 may further include transforming the information into useful medical information 116. For example, the medical information may be formatted in accordance with conventions understood by a trained medical professional. The transformation may be based in part on an analysis of the disturbance characteristics. For example, analysis of the data obtained by sensors may be transformed by computer algorithms or other computational methods to provide evidence of an abnormal heart rate, a focal decrease in energy emission by the heart following a heart attack or other medical conditions involving muscular activity or neurological activity.

In block 1106, the medical information 116 may be provided to one or more of a display 118 and a database 120. The display 118 may be viewable in the study room, so that healthcare professionals attending to the study may have real-time information about the medical conditions. The database 120 may be an electronic medical records database that stores an electronic medical record of the patient. In embodiments, the medical information 116 may be provided to the display 118 and/or database 120 over a network 122, although the network 122 is not necessary and may be omitted.

In embodiments, the processing unit 106 may be any computer or processor-based device capable of performing the functions described herein. Examples of the processing unit 106 may include a server, a mainframe computer, a personal computer, a desktop computer, a laptop computer, a mobile computer, a handheld portable computer, a digital assistant, a personal digital assistant, a cellular phone, a mobile phone, a smartphone, a pager, a digital tablet, an Internet appliance, any other processor-based device, or combinations thereof. The processing unit 106 may be a single integrated unit as shown, or a number of such units. The processing unit 106 may analyze data independently based on algorithms, computer modeling or other forms of analysis. The processing unit 106 may allow user input such as demographic information, patient height, patient weight, and rotation of actuators 225 to optimize the electromagnetic field 110 and adjust the information for the display 118. Manual adjustment of the components of the electromagnetic field generating unit 102 may be permitted. The processing unit 106 may allow user input from devices such as a computer mouse, keyboard, touch-screen display, remote device or other devices. The processing unit 106 may have a user display such as a computer monitor, LED display or other device. The processing unit 106 may also allow voice commands or vocalize settings to the user.

The processing unit 106 may include a processor and a memory. The memory can be coupled to, or in communication with, the processor. The memory may store computer-executable program instructions that, when executed by the processor, cause the processor to perform the steps described herein. In embodiments, computer-executable program instructions stored in a memory of the processing unit 106 may include a medical conditions module 108, which may execute an embodiment of the method. The memory may comprise any computer-readable medium, such as a random-access memory (“RAM”), a read-only memory (“ROM”), or a removable storage device, among others or combinations thereof. The processing unit 106 also may include one or more input/output interfaces (“I/O interfaces”) that facilitate communication with other components of the system 100, including user devices such as a keyboard or a mouse, among others.

The processing unit 106 may communicate with the network 122 via a signal, such as a wired communication signal or a wireless frequency signal. The network 122 may be, for example, the Internet, a local area network (LAN), a wide area network (WAN), a public switched telephone network, or a wireless communications network capable of transmitting voice, data, image, and multimedia signals, among others or combinations thereof. It should be noted that the network 122 is provided by way of example. In embodiments, the network 122 may be eliminated completely and the processing unit 106 may communicate with the display 118 or database 120 directly.

The display 118 may include any display suited for displaying data. Examples of the display 118 can include, but are not limited to, a monitor, a television, or any display in communication with a personal computer or other computing device, an integrated screen or display associated with a personal digital assistant, a cellular phone, a mobile phone, a smartphone, a laptop computer, or other computing device, or a combination of a projecting device and screen, among others. In embodiments, the display 118 may be a single display or multiple displays in a variety of patient care locations. Remote monitoring of the medical information may be made available via the Internet, an Intranet, modem or other telecommunications device. User interfaces may be created using voice commands. Audio displays may be created using vocalizations of data points. Computer-executable program instructions stored in memory may include a display device driver application program, or a display device engine or module. The display device engine or module may be adapted to implement a set of instructions to convert data to a suitable format for display. In embodiments, the display 118 may receive a signal from the display device engine associated with the processing unit 106 and may output medical information onto a screen. The computational tasks associated with rendering a graphical image may be performed by the processing unit 106 or by any other component.

The database 120 may be an electronic medical record database, which may store an electronic medical record of the patient. The processing unit 106 may transmit the medical information 116 to the database 120 over the network 122, although other configurations are possible.

Embodiments of the systems and methods described above may facilitate detecting medical conditions such as congestive heart failure, muscle injury, neurological dysfunction or other medical conditions. A sensing device suited for detecting an electromagnetic field may be associated with a patient, and the patient may be positioned on a bed, chair, or other container. The bed, chair, or other container may create an electromagnetic field that emanates through the patient, and muscle contractions or neurological activity of the patient may create disturbances in the electromagnetic field. These disturbances may be detected by the sensing device and processed by the processing device. The processing device may transform the disturbances into useful medical information regarding the patient's medical condition. The useful medical information may be displayed on a display or stored in a database. The systems and methods may permit detecting changes in electromagnetic energy emissions by organs of the body when conventional methods may be ineffective. The systems and methods also may facilitate detecting muscle contractions and/or neurological activity in obese patients. Thus, the treating provider may have access to medical information that is currently not available. The systems and methods may be used alone throughout the study, or the systems and methods may be combined with conventional methods, for redundancy.

The systems and methods disclosed herein are described with reference to a human patient, although the systems and methods may be employed with reference to other animals, mammalian or otherwise. A veterinarian or other suitable professional may adapt the systems and methods described above either separately or in combination with existing methods for the study of medical conditions for such animals.

It also should be noted that the block diagrams illustrated in the figures may represent functional components, which may be moved, altered, eliminated, or combined with other components when implemented.

While particular embodiments of systems and methods for detecting medical conditions via electromagnetic field disturbances have been disclosed in detail in the foregoing description and figures for purposes of example, those skilled in the art will understand that variations and modifications may be made without departing from the scope of the disclosure. All such variations and modifications are intended to be included within the scope of the present disclosure, as protected by the following claims and the equivalents thereof.

At least the following is claimed:

1. A system for detecting a heart condition in a patient, the system comprising:
an electromagnetic field generating unit operable to create an electromagnetic field about the patient;
an electromagnetic field sensing unit operable to detect a disturbance in the electromagnetic field; and
a processing unit operable to process the disturbance in the electromagnetic field to extract at least one of: a heart condition, a muscular condition, or a neurological condition.

2. The system of claim 1, wherein the electromagnetic field generating unit comprises:
one or more electromagnets; and
a field generator operable to cause the electromagnets to produce an electromagnetic field.

3. The system of claim 2, wherein the field generator is operable to provide a current to the electromagnets.

4. The system of claim 2, wherein the field generator comprises a direct current source and a pulse width modulator.

5. The system of claim 2, wherein the electromagnets are positioned in at least one of a bed or a chair.

6. The system of claim 2, wherein the electromagnetic field generating unit further comprises an actuator operable to move or rotate at least one electromagnet.

7. The system of claim 6, wherein the processing unit is further operable to control the actuator in response to information provided by the electromagnetic field sensing unit.

8. The system of claim 1, wherein the electromagnetic field sensing unit comprises one or more electromagnetic sensors.

9. The system of claim 1, wherein the electromagnetic field sensing unit comprises:
a vest device adapted for positioning about an upper body region of the patient; and
one or more electromagnetic sensors positioned within the vest device.

10. The system of claim 1, wherein the electromagnetic field sensing unit comprises:
a cap device adapted for positioning on a head of the patient; and
one or more electromagnetic sensors positioned within the cap device.

11. The system of claim 1, wherein the electromagnetic field sensing unit comprises:
a sleeve device or abdominal binder adapted for positioning on an arm or leg of the patient; and
one or more electromagnetic sensors positioned within the sleeve device.

12. The system of claim 1, wherein the processing unit is further operable to:
receive electromagnetic field information from the electromagnetic field generating unit; and
receive disturbance information from the electromagnetic field sensing unit.

13. The system of claim 12, wherein the processing unit is further operable to process the electromagnetic field information and the disturbance information to identify a cause of the disturbance in the electromagnetic field.

14. The system of claim 13, wherein the processing unit is further operable to identify the cause of the disturbance as one or more of the following: a cardiac condition, a neurological condition, or a muscular condition.

15. The system of claim 14, wherein the processing unit is further operable to filter disturbances attributable to other causes.

16. The system of claim 1, wherein the processing unit is operable to extract a medical condition by analyzing one or more disturbance characteristics of the disturbance, the disturbance characteristics including one or more of the following: a magnitude of the disturbance, a location of the disturbance, a duration of the disturbance, and a repetition of the disturbance.

17. The system of claim 16, wherein:
the system further comprises at least one component selected from the group consisting of: a display and an electronic medical records database; and
the processing unit is operable to transmit the medical condition to the at least one component.

18. A method of detecting heart conditions via electromagnetic field disturbances, the method comprising:
receiving electromagnetic field information and disturbance information;
processing the electromagnetic field information and the disturbance information to identify medical information; and
transmitting the medical information to one or more of the following: a display and a database.

19. The method of claim 18, wherein the electromagnetic field sensing unit comprises a vest device, a cap device, or a sleeve device, an abdominal binder device wherein one or more electromagnetic sensors are positioned within the vest device, the cap device, or the sleeve device.

20. A system for detecting a medical condition in a patient, the system comprising:
an electromagnetic field generating unit operable to create an electromagnetic field about the patient;
an electromagnetic field sensing unit operable to detect a disturbance in the electromagnetic field; and
a processing unit operable to:
receive electromagnetic field information from the electromagnetic field generating unit;
receive disturbance information from the electromagnetic field sensing unit; and
process the electromagnetic field information and the disturbance information to identify a cause of the disturbance as one or more of the following: a cardiac condition, a neurological condition, or a muscular condition.
